(19) United States
(12) Patent Application Publication
(10) Pub. No.: US 2021/0353082 A1
(43) Pub. Date: Nov. 18, 2021

(54) NOISE CANCELLATION PILLOW AND PILLOW COMBINATION THEREOF

(71) Applicant: SENGLED OPTOELECTRONICS CO., LTD

(72) Inventors: Xinwang LIN, Tongxiang (CN); Chaoqun SUN, Tongxiang (CN); Jinxiang SHEN, Tongxiang (CN)

(21) Appl. No.: 16/328,574

(22) PCT Filed: Aug. 18, 2017
(86) PCT No.: PCT/CN2017/098077

§ 371 Date: Feb. 26, 2019

(30) Foreign Application Priority Data
Aug. 19, 2016 (CN) 201610692429.0

(51) Int. Cl. A47G 9/10; G10K 11/178; A61B 5/00

(52) U.S. Cl. CPC A47G 9/1045; G10K 11/178; A61B 5/4815; A47G 2200/146; G10K 2210/301; A61B 2562/0204; A61B 5/6887

(57) ABSTRACT

A noise cancellation pillow and pillow combination are provided. The pillow includes: a noise collector, a loudspeaker, and a noise cancellation device disposed inside the pillow. The noise collector and the loudspeaker communicate with and are connected to the noise cancellation device, respectively. The noise collector is configured to collect a noise signal generated by a noise source and send the noise signal to the noise cancellation device. The noise cancellation device is configured to receive the noise signal, generate a noise cancellation signal based on the noise signal, and send the noise cancellation signal to the loudspeaker. The loudspeaker is configured to receive the noise cancellation signal sent by the noise cancellation device and radiate the noise cancellation signal, such that the noise signal is counteracted. The disclosed noise cancellation pillow and pillow combination may cancel an ambient noise and improve sleep quality of a user.

### CROSS-REFERENCES TO RELATED APPLICATIONS

This application is a national phase entry under 35 U.S.C. § 371 of International Application No. PCT/CN2017/098077, filed on Aug. 18, 2017, which claims the priority of Chinese Patent Application No. 2016101692429.0 filed on Aug. 19, 2016, the entire content of all of which are incorporated herein by reference.

### FIELD OF THE DISCLOSURE

The present disclosure generally relates to the noise cancellation technology and, more particularly, relates to a noise cancellation pillow and a pillow combination thereof.

### BACKGROUND

As the pace of modern society accelerates, people have to deal with more pressure. People need to have a healthy body and mind to better handle such living pressure. The sleep quality of a person often directly impacts the health condition of his or her body, and various factors may affect the sleep quality. For example, factors such as physiological diseases, the mental pressure caused by the fast working and living pace in the cities, and the ambient noise, etc. may all lead to a low sleep quality. In particular, the sleep quality issues caused by a bedmate making noises, such as snoring, is particularly difficult to resolve.

To improve people's sleep quality, a pillow that cancels the ambient noise is desired.

### BRIEF SUMMARY OF THE DISCLOSURE

The present disclosure provides a noise cancellation pillow and a pillow combination thereof. The disclosed noise cancellation pillow and pillow combination are directed towards solving the existing issue of the ambient noise impacting the sleep quality of the people in sleep.

In a first aspect, the present disclosure provides a noise cancellation pillow, comprising: a noise collector, a loudspeaker, and a noise cancellation device that are disposed inside the pillow. The noise collector and the loudspeaker may communicate and be connected to the noise cancellation device, respectively.

The noise collector is configured to collect a noise signal sent out by the noise source and send the noise signal to the noise cancellation device.

The noise cancellation device is configured to receive the noise signal sent by the noise collector, generate a noise cancellation signal corresponding to the noise signal based on the noise signal, and send the noise cancellation signal to the loudspeaker.

The loudspeaker is configured to receive the noise cancellation signal sent by the noise cancellation device, and radiate the noise cancellation signal into the air, such that the noise cancellation signal counteracts the noise signal.

Optionally, the noise cancellation device is specifically configured to; based on the noise signal, generate the noise cancellation signal that has a same frequency, same amplitude, and an opposite phase of the noise signal.

Optionally, the pillow further includes a locating device, and the locating device may communicate and be connected to the noise collector.

The locating device is configured to, based on the noise signal sent by the noise collector, determine position information of a user's head in the pillow, and send the position information to the noise cancellation device.

The noise cancellation device is specifically configured to adjust a frequency, an amplitude, and a phase of the noise cancellation signal based on the position information.

Optionally, the noise collector may include a first noise collector and a second noise collector. The first noise collector and the second noise collector may be connected to the noise cancellation device, respectively.

The first noise collector and the second noise collector are configured to collect a first noise signal and a second noise signal of the noise source, and send the first noise signal and the second noise signal to the noise cancellation device, respectively.

The noise cancellation device is configured to: receive the first noise signal and the second noise signal respectively sent by the first noise collector and the second noise collector; and based on the first noise signal and the second noise signal, generate a first noise cancellation signal corresponding to the first noise signal and generate a second noise cancellation signal corresponding to the second noise signal.

Optionally, the loudspeaker includes a first loudspeaker and a second loudspeaker. The first loudspeaker and the second loudspeaker may communicate with and be connected to the noise cancellation device, respectively.

The first loudspeaker is configured to: receive the first noise cancellation signal sent by the noise cancellation device, and radiate the first noise cancellation signal into the air.

The second loudspeaker is configured to: receive the second noise cancellation signal sent by the noise cancellation device and radiate the second noise cancellation signal into the air.

Optionally, the first noise collector and the second noise collector may be located on two sides of a central line of the pillow, and be configured to be axially symmetric with respect to the central line of the pillow. The first loudspeaker and the second loudspeaker may be located on two sides of the central line of the pillow, and be configured to be axially symmetric with respect to the central line of the pillow.

Optionally, the pillow may further include: a sleep quality monitor and a wireless transmitter that are disposed in the pillow. The sleep quality monitor may communicate and be connected to the wireless transmitter.

The sleep quality monitor is configured to monitor a heart rate, breathing information, and head rotation frequency information of a user that sleeps on the pillow, and send the heart rate, the breathing information, and the head rotation frequency information to a user terminal via a wireless module.

In a second aspect, the present disclosure further provides a pillow combination, comprising a first pillow and a second pillow. The first pillow may be any one of the aforementioned pillows. An error noise collector may be disposed in the second pillow, and the error noise collector may communicate and be connected to the noise cancellation device in the first pillow.

The error noise collector is configured to collect a residual noise signal on two sides of the second pillow, and send the residual noise signal to the noise cancellation device.

The noise cancellation device is configured to receive the residual noise signal, and based on the residual noise signal, adjust the noise cancellation signal.

Optionally, the pillow combination may further include a feedback device disposed in the first pillow or the second pillow. The feedback device may communicate and be connected to the error noise collector. The feedback device is configured to: receive the residual noise signal sent by the error noise collector, and determine whether the residual noise signal is smaller than or equal to a preset reference noise signal.

The error noise collector is specifically configured to, when the feedback device determines that the residual noise signal is greater than the preset reference noise signal, send the residual noise signal to the noise cancellation device.

Optionally, the error noise collector may include a first error noise collector and a second error noise collector. The first error noise collector and the second error noise collector may be located on two sides of a central line of the second pillow, and may be configured to be axially symmetric with respect to the central line of the second pillow.

In a third aspect, the present disclosure further provides a pillow combination, comprising a first pillow and a second pillow. The first pillow comprises a noise collector, a loudspeaker, and a noise cancellation device that are disposed in the first pillow; and an error noise collector is disposed in the second pillow, and the error noise collector is connected to the noise cancellation device in the first pillow. The noise collector and the loudspeaker are connected to the noise cancellation device, respectively. The noise collector is configured to collect a noise signal sent by a noise source and send the noise signal to the noise cancellation device, the noise cancellation device is configured to receive the noise signal sent by the noise collector, generate a noise cancellation signal corresponding to the noise signal based on the noise signal, and send the noise cancellation signal to the loudspeaker, the loudspeaker is configured to receive the noise cancellation signal sent by the noise cancellation device, and radiate the noise cancellation signal, thereby counteracting the noise signal, the error noise collector is configured to collect a residual noise signal on two sides of the second pillow and send the residual noise signal to the noise cancellation device, and the noise cancellation device is further configured to receive the residual noise signal, and based on the residual noise signal, adjust the noise cancellation signal.

The first pillow further may include a locating device. The locating device may be connected to the noise collector. The locating device may be configured to, based on the noise signal sent by the noise collector, determine position information of a head of a user on the pillow, and send the position information to the noise cancellation device; and the noise cancellation device is configured to adjust a frequency, an amplitude, and a phase of the noise cancellation signal based on the position information.

The noise collector includes a first noise collector and a second noise collector, and the first noise collector and the second noise collector are connected to the noise cancellation device, respectively. The first noise collector and the second noise collector may be configured to collect a first noise signal and a second noise signal of the noise source, respectively, and send the first noise signal and the second noise signal of the noise source to the noise cancellation device, respectively. The noise cancellation device may be configured to: receive the first noise signal and the second noise signal respectively sent by the first noise collector and the second noise collector, and based on the first noise signal and the second noise signal, generate a first noise cancellation signal corresponding to the first noise signal and a second noise cancellation signal corresponding to the second noise signal.

The loudspeaker may include a first loudspeaker and a second loudspeaker, and the first loudspeaker and the second loudspeaker are connected to the noise cancellation device, respectively. The first loudspeaker may be configured to receive the first noise cancellation signal sent by the noise cancellation device and radiate the first noise cancellation signal; and the second loudspeaker may be configured to receive the second noise cancellation signal sent by the noise cancellation device and radiate the second noise cancellation signal.

The first noise collector and the second noise collector may be located on two sides of a central line of the pillow and are configured to be axially symmetric with respect to the central line of the pillow; and the first loudspeaker and the second loudspeaker are located on two sides of the central line of the pillow and are configured to be axially symmetric with respect to the central line of the pillow.

The first pillow further may include a sleep quality monitor and a wireless transmitter that are disposed inside the pillow. The sleep quality monitor may be connected to the wireless transmitter; and the sleep quality monitor may be configured to monitor a heart rate, breathing information, and head rotation frequency information of a user that sleeps on the pillow, and send the heart rate, the breathing information, and the head rotation frequency information to a user terminal via a wireless module.

The pillow combination may further include a feedback device disposed in the first pillow or the second pillow. The feedback device may be connected to the error noise collector, and the feedback device may be configured to: receive the residual noise signal sent by the error noise collector and determine whether the residual noise signal is smaller than or equal to a preset reference noise signal, and the error noise collector is configured to: when the feedback device determines that the residual noise signal is greater than the preset reference noise signal, send the residual noise signal to the noise cancellation device.

The error noise collector may include a first error noise collector and a second error noise collector, the first error noise collector and the second error noise collector are located on two sides of a central line of the second pillow and are configured to be axially symmetric with respect to the central line of the second pillow.

Embodiments of the present disclosure provide a noise cancellation pillow. By using the disclosed noise cancellation pillow, the noise signal of the noise source is collected via the noise collector, the noise cancellation signal is generated based on the noise signal via the noise cancellation device, and the noise cancellation signal is radiated into the air via a loudspeaker to counteract the noise signal, such that the ambient noise may be cancelled. Accordingly, during a process when the user uses the pillow, the user would not be disturbed by the noise, and the sleep quality of the user may be improved. Embodiments of the present disclosure further provide a pillow combination. By using the pillow combination provided by embodiments of the present disclosure, a noise cancellation process on the noise sent by a first user that uses the first pillow is performed via the noise cancellation device in the first pillow, the residual noise signal received by the ear of the user that uses the second pillow is collected, and the residual noise signal is further counteracted. Accordingly, the noise signals eventually received by the second user that uses the second pillow may achieve an expected noise cancellation effect, such that the sleep quality of the second user may be improved.

### BRIEF DESCRIPTION OF THE DRAWINGS

To more clearly illustrate the technical solutions in embodiments of the present invention or the prior art, the accompanying drawings needed for describing the disclosed embodiments or the prior art are briefly introduced hereinafter. Obviously, the accompanying drawings in the following descriptions are merely some embodiments of the present invention. Based on such accompanying drawings, other drawings may be obtainable by those ordinarily skilled in the art without creative effort.

FIG. 1 illustrates a structural schematic view of a noise cancellation pillow consistent with various disclosed embodiments;

FIG. 2 illustrates another structural schematic view of a noise cancellation pillow consistent with various disclosed embodiments; and

FIG. 3 illustrates a structural schematic view of a pillow combination consistent with various disclosed embodiments.

### DETAILED DESCRIPTION

To make the objective, technical solutions and advantages of embodiment of the present disclosure more clearly, technical solutions in embodiments of the present disclosure will be described in a clear and complete manner hereinafter with reference to the accompanying drawings in the present disclosure. Obviously, the embodiments described herein are merely a part of, but not entire embodiments of the present disclosure. Based on the embodiments of the present disclosure, other embodiments obtainable by those ordinarily skilled in the art without creative effort shall all fall within the protection scope of the present disclosure.

Embodiments of the present disclosure provide a noise cancellation pillow and a pillow combination. The pillow and the pillow combination may be configured to cancel ambient noise when a user is under rest, such that the sleep quality of the user may improved.

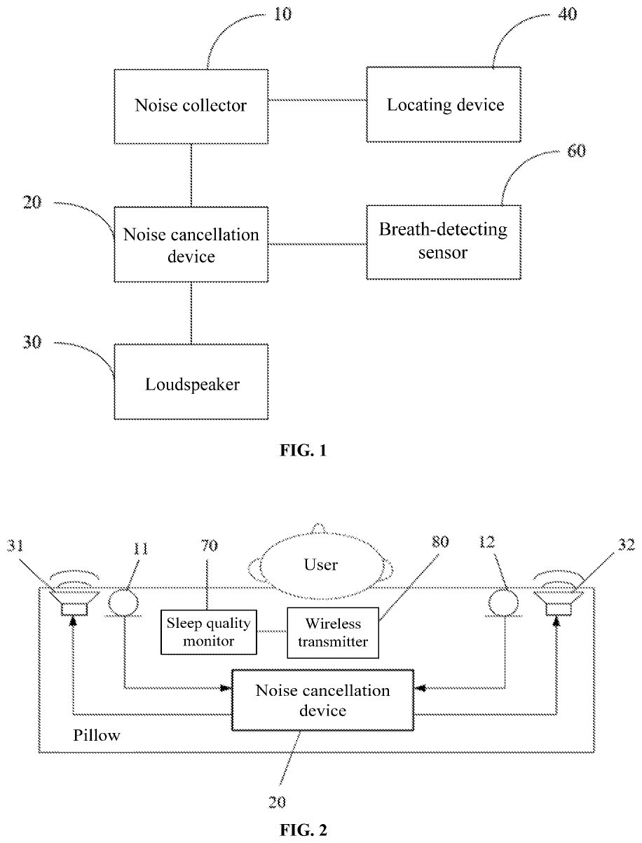

FIG. 1 illustrates a structural schematic view of a pillow that cancels noise consistent with various disclosed embodiments.

Referring to FIG. 1, noise cancellation pillow provided by embodiments of the present disclosure may include the following components.

A noise collector 10, a noise cancellation device 20, and a loudspeaker 30 disposed in the pillow. The noise collector 10 and the loudspeaker 30 may communicate and be connected to the noise cancellation device 20.

The noise collector 10 may be configured to collect a noise signal generated by a noise source and send the noise signal to the noise cancellation device 20.

The noise cancellation device 20 may be configured to receive the noise signal sent by the noise collector 10, generate a noise cancellation signal corresponding to the noise signal based on the noise signal, and send the noise cancellation signal to the loudspeaker 30.

The loudspeaker 30 may be configured to receive the noise cancellation signal sent by the noise cancellation device, and radiate the noise cancellation signal up to the air, thereby offsetting the noise signal.

More specifically, the noise cancellation device 20 may be configured to, based on noise signal, generate the noise cancellation signal that has the same frequency and amplitude as the noise signal but a different phase.

The noise cancellation device 20 may specifically include a digital signal processor and an analog signal processor. The analog signal processor may be configured to pre-amplify the collected noise signal and output the pre-amplified noise signal to the digital signal processor. The digital signal processor may be configured to perform A/D conversion on the pre-amplified noise signal, and based on the noise signal after A/D conversion, generate a noise cancellation signal that has the same frequency and amplitude as the noise signal but a different phase. Further, after performing A/D conversion on the noise cancellation signal, the digital signal processor may be configured to perform audio amplifying on the noise cancellation signal after A/D conversion and output the enlarged noise cancellation signal to the loudspeaker 30.

By using the noise cancellation pillow provided by embodiments of the present disclosure, the noise collector may collect the noise signal of the noise source, the noise cancellation device may generate a noise cancellation signal based on the noise signal, and the loudspeaker may radiate the noise cancellation signal up to the air to counteract the noise signal, thereby cancelling the ambient noise. Accordingly, during the process of the user using the disclosed pillow for a rest, the user would not be disturbed by the noise, such that the sleep quality of the user may be improved.

Further, based on the aforementioned embodiments, the pillow may further include a locating device 40. The locating device 40 may communicate with and be connected to the noise collector 10.

The locating device 40 may be configured to, based on the noise signal sent by the noise collector 10, determine the position information of the user's head in the pillow and send the position information to the noise cancellation device 20.

Correspondingly, the noise cancellation device may be configured to, based on the position information, adjust the frequency, amplitude, and phase of the noise cancellation signal.

By locating the position of the user's head in the pillow via the locating device, even if the user rotates or moves the head during sleep such that the user's head is not in the central position of the pillow, the disclosed noise cancellation pillow may still correspondingly adjust the frequency, amplitude, and/or phase of the noise cancellation signal based on the located position of the user head, thereby improving the effect of noise cancellation.

Further, based on the aforementioned embodiments, the pillow may further include a breath-detecting sensor 60. The breath-detecting sensor 60 may communicate with and be connected to the noise cancellation device 20. The breath-detecting sensor 60 may be configured to detect the breathing rhythm, frequency and depth information of the user that sleeps on the pillow, and further feedback the breathing rhythm, frequency and depth information of the user to the noise cancellation device 20. Correspondingly, the noise cancellation device may further be configured to, based on the breathing rhythm, frequency and depth information of the user to adjust the frequency, the amplitude and/or the phase of the noise cancellation signal, thereby improving the effect of noise cancellation.

FIG. 2 illustrates another structural schematic view of a noise cancellation pillow consistent with various disclosed embodiments.

Referring to FIG. 2, further based on the aforementioned embodiments, the noise collector 10 may include a first noise collector 11 and a second noise collector 12. The first noise collector 11 and the second noise collector 12 may be connected to the noise cancellation device 20, respectively. More specifically, the first noise collector 11 and the second noise collector 12 may be coupled to the noise cancellation device 20 via wired connection or wireless connection.

The first noise collector 11 and the second noise collector 12 may be configured to collect a first noise signal and a second noise signal of the noise source, respectively, and send the first noise signal and the second noise signal to the noise cancellation device 20, respectively.

Correspondingly, the noise cancellation device 20 may be configured to: receive the first noise signal and the second noise signal respectively sent by the first noise collector 11 and the second noise collector 12; and based on the first noise signal and the second noise signal, generate a first noise cancellation signal corresponding to the first noise signal and a second noise cancellation signal corresponding to the second noise signal.

Further, the loudspeaker 30 may include a first loudspeaker 31 and a second loudspeaker 32. The first loudspeaker 31 and the second loudspeaker 32 may communicate with and be connected to the noise cancellation device 20, respectively. More specifically, the first loudspeaker 31 and the second loudspeaker 32 may be connected to the noise cancellation device 20 via wired connection or wireless connection.

The first loudspeaker 31 may be configured to: receive the first noise cancellation signal sent by the noise cancellation device 20, and radiate the first noise cancellation signal up to the air.

The second loudspeaker 32 may be configured to: receive the second noise cancellation signal sent by the noise cancellation device 20, and radiate the second noise cancellation signal up to the air.

The first noise cancellation signal and the second noise cancellation signal may be combined to form a third noise cancellation signal that counteracts the noise signal of the noise source. Accordingly, a silent zone may be formed to reduce the impact of the noise generated by the person that snores on people nearby, thereby improving the user's sleep quality.

Further, based on the aforementioned embodiments, the first noise collector 11 and the second noise collector 12 may be located on two sides of the central line of the pillow, and may be configured to be axially symmetrically with respect to the central line of the pillow.

By configuring the first noise collector 11 and the second noise collector 12 to be axially symmetric with respect to the central line of the pillow, and by configuring the first loudspeaker 31 and the second loudspeaker 32 to be axially symmetric with respect to the central line of the pillow, the noise signal radiated by the noise source may be collected relatively comprehensively, such that the noise signal may be counteracted precisely.

Further, based on the aforementioned embodiments, the pillow may further include a sleep quality monitor 70 and a wireless transmitter 80 that are disposed inside the pillow. The sleep quality monitor 70 and the wireless transmitter 80 may communicate and be connected.

The sleep quality monitor 70 may be configured to monitor a heart rate, breathing information, and head rotation frequency information of the user that sleeps on the pillow, and send the heart rate, the breathing information, and the head rotation frequency information to a user terminal via the wireless transmitter 80, thereby forming a sleep quality report. The user may carry out corresponding health care actions based on such sleep quality report. In some embodiments, the sleep quality monitor 70 may include a display and a control mechanism. A user may check the sleep quality data on the display, and use the control mechanism to select the sleep quality indicators to be monitored and relevant indicator values. For example, a user my select to monitor the heart rate, and set a range of acceptable heart rate, such as from 60 times/minute-100 times/minute. When a user's heart rate falls out of the acceptable range, the sleep quality monitor 70 may display a warning sign or a warning message.

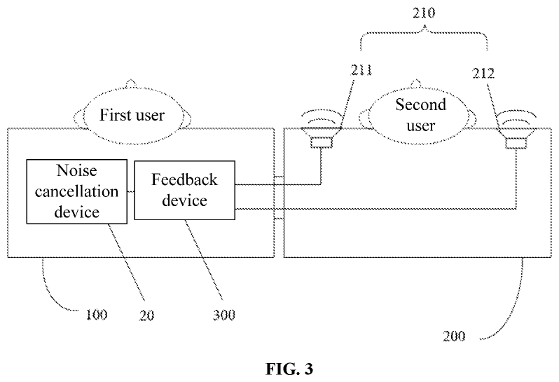

FIG. 3 illustrates a structural schematic view of a pillow combination consistent with various disclosed embodiments.

Referring to FIG. 3, the disclosed pillow combination may include a first pillow 100 and a second pillow 200. The first pillow 100 may be any pillow according to the aforementioned embodiments. Further, an error noise collector 210 may be disposed in the second pillow 200. The error noise collector 210 may communicate with and be connected to the noise cancellation device 20 in the first pillow 100.

The error noise collector 210 may be configured to collect the residual noise signal on two sides of the second pillow 200, and send the residual noise signal to the noise cancellation device 20.

The noise cancellation device 20 may be configured to receive the residual noise signal, and adjust the noise cancellation signal based on the residual noise signal.

When in use, the second pillow 200 and the first pillow 100 may be arranged side by side. Further, the second pillow 200 may be connected to the first pillow 100 or may be individually separated.

More specifically, the error noise collector 210 may include a first error noise collector 211 and a second error noise collector 212. The first error noise collector 211 and the second error noise collector 212 may be located on two sides of the central line of the second pillow 200 and may be axially symmetric with respect to the central line of the second pillow 200.

By configuring the first error noise collector 211 and the second noise collector 212 to be axially symmetric with respect to the central line of the second pillow and by respectively configuring the first error noise collector 211 and the second error noise collector 212 to be as close to the ears of the user that sleeps on the second pillow 200 as possible, the residual noise to be heard by the second user that uses the second pillow 200 for sleep may be accurately acquired. Accordingly, the generation of a counteract signal directed towards the residual noise may be ensured via the noise cancellation device 20, such that the noise cancellation effect may be further improved.

Further, based on the aforementioned embodiments, the pillow combination may further include: a feedback device 300 disposed in the first pillow 100 or the second pillow 200. The feedback device 300 may communicate with and be connected to the error noise collector 210. The feedback device 300 may be configured to: receive the residual noise signal sent by the error noise collector 210, and determine whether the residual noise signal is smaller than or equal to a preset reference noise signal.

The error noise collector 210 may be specifically configured to: when the feedback device 300 determines that the residual noise signal is greater than the preset reference noise signal, send the residual noise signal to the noise cancellation device 20. The noise cancellation device 20 may then continue to perform a counteracting process towards the residual noise signal using the aforementioned method.

The pillow combination provided by embodiments of the present disclosure may perform a noise cancellation process on the noise generated by the first user that uses the first pillow 100 via the noise cancellation device 20 in the first pillow 100. Further, by collecting the residual noise signal received by the ears of the user that uses the second pillow 200, the disclosed pillow combination may further counteract the residual noise signal, such that the noise signal eventually received by the second user that uses the second pillow 200 may achieve a desired noise cancellation effect. Accordingly, the sleep quality of the second user may be improved.

Those ordinarily skilled in the relevant art may understand that: the implementation of all or a part of steps in each aforementioned method embodiment may be fulfilled via hardware related to the program command. The above-described program may be stored in a computer readable storage medium. When the program is executed, the steps of each aforementioned method embodiment may be executed. Further, the aforementioned storage media may include media that stores various kinds of program codes such as ROM, RAM, magnetic disc, or optical disc.

Finally it should be illustrated that, those embodiments above are used to illustrate technical solutions of the present disclosure, but not to limit the scope of the disclosure. Though, referring to previous embodiments, the present disclosure is illustrated in details, those ordinarily skilled in the art may still understand that technical solutions in the previous embodiments may be modified, or either partial or entire technical characteristics may be equally exchanged. Via such modification or exchange, the nature of the corresponding technical solutions will not depart from the scope of the present disclosure.

What is claimed is:

1. A noise cancellation pillow, comprising a noise collector, a loudspeaker, and a noise cancellation device that are disposed in the pillow, wherein:
the noise collector and the loudspeaker are connected to the noise cancellation device, respectively;
the noise collector is configured to collect a noise signal sent by a noise source and send the noise signal to the noise cancellation device;
the noise cancellation device is configured to receive the noise signal sent by the noise collector, generate a noise cancellation signal corresponding to the noise signal based on the noise signal, and send the noise cancellation signal to the loudspeaker; and
the loudspeaker is configured to receive the noise cancellation signal sent by the noise cancellation device, and radiate the noise cancellation signal, thereby counteracting the noise signal.

2. The pillow according to claim 1, wherein:
the noise cancellation device is configured to, based on the noise signal, generate the noise cancellation signal that has a same frequency, same amplitude but an opposite phase with respect to the noise signal.

3. The pillow according to claim 1, further comprising a locating device, wherein:
the locating device is connected to the noise collector;
the locating device is configured to, based on the noise signal sent by the noise collector, determine position information of a head of a user on the pillow, and send the position information to the noise cancellation device; and
the noise cancellation device is configured to adjust a frequency, amplitude, and a phase of the noise cancellation signal based on the position information.

4. The pillow according to claim 1, wherein:
the noise collector includes a first noise collector and a second noise collector, and the first noise collector and the second noise collector are connected to the noise cancellation device, respectively;
the first noise collector and the second noise collector are configured to collect a first noise signal and a second noise signal of the noise source, respectively, and send the first noise signal and the second noise signal of the noise source to the noise cancellation device, respectively; and
the noise cancellation device is configured to:
receive the first noise signal and the second noise signal respectively sent by the first noise collector and the second noise collector, and
based on the first noise signal and the second noise signal, generate a first noise cancellation signal corresponding to the first noise signal and a second noise cancellation signal corresponding to the second noise signal.

5. The pillow according to claim 4, wherein:
the loudspeaker includes a first loudspeaker and a second loudspeaker, and the first loudspeaker and the second loudspeaker are connected to the noise cancellation device, respectively;
the first loudspeaker is configured to receive the first noise cancellation signal sent by the noise cancellation device and radiate the first noise cancellation signal; and
the second loudspeaker is configured to receive the second noise cancellation signal sent by the noise cancellation device and radiate the second noise cancellation signal.

6. The pillow according to claim 5, wherein:
the first noise collector and the second noise collector are located on two sides of a central line of the pillow and are configured to be axially symmetric with respect to the central line of the pillow; and
the first loudspeaker and the second loudspeaker are located on two sides of the central line of the pillow and are configured to be axially symmetric with respect to the central line of the pillow.

7. The pillow according to claim 1, further comprising a sleep quality monitor and a wireless transmitter that are disposed inside the pillow, wherein:
the sleep quality monitor is connected to the wireless transmitter; and
the sleep quality monitor is configured to monitor a heart rate, breathing information, and head rotation frequency information of a user that sleeps on the pillow, and send the heart rate, the breathing information, and the head rotation frequency information to a user terminal via the wireless transmitter.

8. A pillow combination, comprising a first pillow and a second pillow, wherein:
the first pillow is a pillow described in claim 2;
an error noise collector is disposed in the second pillow, and the error noise collector is connected to the noise cancellation device in the first pillow;
the error noise collector is configured to collect a residual noise signal on two sides of the second pillow and send the residual noise signal to the noise cancellation device; and
the noise cancellation device is configured to receive the residual noise signal, and based on the residual noise signal, adjust the noise cancellation signal.

9. The pillow combination according to claim 8, further comprising a feedback device disposed in the first pillow or the second pillow, wherein:
the feedback device is connected to the error noise collector, and
the feedback device is configured to:
receive the residual noise signal sent by the error noise collector and determine whether the residual noise signal is smaller than or equal to a preset reference noise signal, and
the error noise collector is configured to: when the feedback device determines that the residual noise signal is greater than the preset reference noise signal, send the residual noise signal to the noise cancellation device.

10. The pillow combination according to claim 8, wherein:
the error noise collector includes a first error noise collector and a second error noise collector, the first error noise collector and the second error noise collector are located on two sides of a central line of the second pillow and are configured to be axially symmetric with respect to the central line of the second pillow.

11. A pillow combination, comprising a first pillow and a second pillow, wherein:
the first pillow comprises a noise collector, a loudspeaker, and a noise cancellation device that are disposed in the first pillow; and
an error noise collector is disposed in the second pillow, and the error noise collector is connected to the noise cancellation device in the first pillow,
wherein the noise collector and the loudspeaker are connected to the noise cancellation device, respectively,
the noise collector is configured to collect a noise signal sent by a noise source and send the noise signal to the noise cancellation device,
the noise cancellation device is configured to receive the noise signal sent by the noise collector, generate a noise cancellation signal corresponding to the noise signal based on the noise signal, and send the noise cancellation signal to the loudspeaker,
the loudspeaker is configured to receive the noise cancellation signal sent by the noise cancellation device, and radiate the noise cancellation signal, thereby counteracting the noise signal,
the error noise collector is configured to collect a residual noise signal on two sides of the second pillow and send the residual noise signal to the noise cancellation device, and
the noise cancellation device is further configured to receive the residual noise signal, and based on the residual noise signal, adjust the noise cancellation signal.

12. The pillow combination according to claim 11, wherein:
the first pillow further comprises a locating device;
the locating device is connected to the noise collector;
the locating device is configured to, based on the noise signal sent by the noise collector, determine position information of a head of a user on the pillow, and send the position information to the noise cancellation device; and
the noise cancellation device is configured to adjust a frequency, amplitude, and a phase of the noise cancellation signal based on the position information.

13. The pillow combination according to claim 11, wherein:
the noise collector includes a first noise collector and a second noise collector, and the first noise collector and the second noise collector are connected to the noise cancellation device, respectively;
the first noise collector and the second noise collector are configured to collect a first noise signal and a second noise signal of the noise source, respectively, and send the first noise signal and the second noise signal of the noise source to the noise cancellation device, respectively; and
the noise cancellation device is configured to:
receive the first noise signal and the second noise signal respectively sent by the first noise collector and the second noise collector; and
based on the first noise signal and the second noise signal, generate a first noise cancellation signal corresponding to the first noise signal and a second noise cancellation signal corresponding to the second noise signal.

14. The pillow combination according to claim 13, wherein:
the loudspeaker includes a first loudspeaker and a second loudspeaker, and the first loudspeaker and the second loudspeaker are connected to the noise cancellation device, respectively;
the first loudspeaker is configured to receive the first noise cancellation signal sent by the noise cancellation device and radiate the first noise cancellation signal; and
the second loudspeaker is configured to receive the second noise cancellation signal sent by the noise cancellation device and radiate the second noise cancellation signal.

15. The pillow combination according to claim 14, wherein:
the first noise collector and the second noise collector are located on two sides of a central line of the pillow and are configured to be axially symmetric with respect to the central line of the pillow; and
the first loudspeaker and the second loudspeaker are located on two sides of the central line of the pillow and are configured to be axially symmetric with respect to the central line of the pillow.

16. The pillow combination according to claim 11, wherein:
the first pillow further comprises a sleep quality monitor and a wireless transmitter that are disposed inside the pillow;
the sleep quality monitor is connected to the wireless transmitter; and
the sleep quality monitor is configured to monitor a heart rate, breathing information, and head rotation frequency information of a user that sleeps on the pillow, and send the heart rate, the breathing information, and the head rotation frequency information to a user terminal via the wireless transmitter.

17. The pillow combination according to claim 11, further comprising a feedback device disposed in the first pillow or the second pillow, wherein:
the feedback device is connected to the error noise collector, and
the feedback device is configured to:
receive the residual noise signal sent by the error noise collector and determine whether the residual noise signal is smaller than or equal to a preset reference noise signal; and
the error noise collector is configured to: when the feedback device determines that the residual noise signal is greater than the preset reference noise signal, send the residual noise signal to the noise cancellation device.

18. The pillow combination according to claim 11, wherein:
the error noise collector includes a first error noise collector and a second error noise collector, the first error noise collector and the second error noise collector are located on two sides of a central line of the second pillow and are configured to be axially symmetric with respect to the central line of the second pillow.
